(19) United States
(12) Patent Application Publication
(10) Pub. No.: US 2017/0173039 A1
(43) Pub. Date: Jun. 22, 2017

(54) COMPOSITIONS AND METHODS TO TREAT ADDICTION

(71) Applicants: YALE UNIVERSITY, New Haven, CT (US); THE UNITED STATES GOVERNMENT REPRESENTED BY THE DEPARTMENT OF VETERANS AFFAIRS, Washington, DC (US)

(72) Inventors: Albert ARIAS, Woodbridge, CT (US); Ismene PETRAKIS, Branford, CT (US); John H. KRYSTAL, Woodbridge, CT (US)

(21) Appl. No.: 15/129,158

(22) PCT Filed: Mar. 26, 2015
(86) PCT No.: PCT/US15/22671

§ 371 Date: Sep. 26, 2016

Related U.S. Application Data
(60) Provisional application No. 61/973,961, filed on Apr. 2, 2014.

(51) Int. Cl. A61K 31/551; A61K 31/4725

(52) U.S. Cl. CPC A61K 31/551; A61K 31/4725; A61K 2300/00

(57) ABSTRACT

The present invention provides compositions and methods comprising talampanel and/or perampanel for treating or preventing an addictive disease or disorder, such as alcoholism, in a subject in need thereof. AMPA receptor (AMPA-Rs) antagonists (i.e., blockers), such as perampanel and talampanel, reduce drug and alcohol seeking behaviors in mammals since activation of glutamatergic AMPA receptors promotes addictive behaviors in mammals.

### CROSS-REFERENCE TO RELATED APPLICATIONS

The present application claims priority to U.S. Provisional Patent Application No. 61/973,961, filed Apr. 2, 2014, the entire disclosure of which is incorporated by reference herein as if set forth herein in its entirety.

### STATEMENT REGARDING FEDERALLY SPONSORED RESEARCH OR DEVELOPMENT

This invention was made with government support under AA017689 awarded by National Institute of Health. The government has certain rights in the invention.

### BACKGROUND OF THE INVENTION

Alcoholism may be viewed as a disease, a drug addiction, a learned response to crisis, a symptom of an underlying psychological or physical disorder, or a combination of these factors. Most approaches to the treatment of alcoholism require the alcoholic person to recognize his/her illness and to abstain from alcohol. Treatment programs then vary according to the accepted definition and theory of cause of alcoholism. Treatment types include combinations of: psychological rehabilitative treatments; organized self-help groups; aversion therapy based on behavior modification; injections of vitamins or hormones, and the use of abstinence-maintaining drugs.

Glutamate, the major excitatory neurotransmitter in the brain, participates in neuroprolific, neurotoxic, neurodegenerative, and neuromodulatory processes. Glutamate acts on numerous receptor sites within the central nervous system including N-methyl-D-aspartate (NMDA), alpha-amino-3-hydroxyl-5-methyl-isoxazole-4-proprionic acid (AMPA), and kainite receptors. While much preclinical and clinical research has drawn attention to NMDA-related glutaminergic transmission in psychopathology, AMPA receptors may have a more significant role than other glutamatergic receptors in glutamate-facilitated dopamine release in the nucleus accumbens shell (NAS).

Glutamatergic efferents from the prefrontal cortex (PFC), amygdala, and hippocampus innervate the cell bodies of neurons in the ventral tegmental area (VTA) and the nucleus accumbens shell (NAS), facilitating dopaminergic transmission in these key areas of the “reward pathway.” Pharmacologic AMPA receptor potentiators (agonists) have been shown in rats to reduce the effects of ethanol intoxication (Alcohol Clin Exp Res. 2008; 32(3):489-97), which may be a desirable effect in itself for some situations, but it reduces protective subjective experiences of alcohol, which results in worsening the risk of alcoholism. Consequently, persons that are less sensitive to the unpleasant (aversive) intoxicating effects of alcohol are thought to be at a greater risk for developing alcoholism (Arch Gen Psychiatry. 2007; 64(9):1069-77).

A very recent study in rats confirmed the theory that enhanced AMPA-R mediated glutamatergic transmission due to neuroadaptation from chronic alcohol exposure is a main component of the drive to drink excessively and to crave alcohol. Successful development of medications for alcohol use disorders, such as naltrexone and topiramate, have been shown to reduce the positive effects of alcohol. However, topiramate which is efficacious for alcoholism, does not consistently reduce cue-elicited craving. Further, some medications, such as memantine, decrease craving but do not reduce drinking. Therefore, a need exists in the art for improved treatments and methods to treat addictive diseases and disorders, especially in the case of alcoholism.

### SUMMARY OF THE INVENTION

The invention includes compositions and methods of treating or preventing an addictive disease or disorder. In one aspect, the invention includes a method of treating or preventing an addictive disease or disorder in a subject in need thereof comprising administering an effective amount of at least one of talampanel and perampanel.

In another aspect, the invention includes a method of decreasing alcohol consumption in a subject in need thereof comprising administering an effective amount of at least one of talampanel and perampanel.

In yet another aspect, the invention includes a composition for treating or preventing an addictive disease or disorder, comprising an effective amount of at least one of talampanel and perampanel.

In still another aspect, the invention includes a composition for treating or preventing an addictive disease or disorder, comprising an effective amount of at least one of talampanel and perampanel, wherein the addictive disease or disorder is selected from the group consisting of alcoholism, alcohol dependence, or alcohol withdrawal, heavy alcohol consumption, excessive alcohol consumption, and combinations thereof.

In various embodiments of the above aspects or any other aspect of the invention delineated herein, the invention includes the addictive disease or disorder comprises heavy alcohol consumption or excessive alcohol consumption. In another embodiment, the addictive disease or disorder is alcoholism, alcohol dependence, or alcohol withdrawal. In such an embodiment, the step of administrating results in decreasing frequency of alcohol consumption. In another embodiment, the step of administrating results in decreasing alcohol consumption compared with before administration. In yet another embodiment, the step of administrating results in decreasing alcohol consumption and increasing abstinence of alcohol consumption.

In another embodiment, the step of administering the effective amount comprises administering about 0.05 mg to about 0.5 g per single dose, less than about 0.80 g daily, or within the range of about 0.001 mg/kg to about 100 mg/kg. In yet another embodiment, the step of administering comprises administration via an oral route.

In one embodiment, the composition further comprises a pharmaceutically acceptable carrier or adjuvant, such as including at least one binder, excipient, diluent, or any combinations thereof. In another embodiment, the composition is formulated for oral administration, such as a liquid suspension, a chewable composition, and an orally disintegrating tablet or capsule composition. In another embodiment, the composition is formulated for delayed-release.

In another embodiment, the effective amount comprises about 2 mg to about 100 mg per single dose. In yet another embodiment, the effective amount comprises less than about 50 mg daily.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or within 10%, 9%, 8%, 7%, 6%, 5%, 4%, 3%, 2%, 1%, 0.5%, 0.1%, 0.05%, or 0.01% of the specified value, as such variations are appropriate to perform the disclosed methods. Unless otherwise clear from context, all numerical values provided herein are modified by the term about.

By “addictive disease or disorder” is meant a primary or chronic disease of brain reward, motivation, memory and related circuitry. Dysfunction in these circuits leads to characteristic biological, psychological, social and spiritual manifestations. This is reflected in an individual pathologically pursuing reward and/or relief by substance use and other behaviors. Addiction is characterized by inability to consistently abstain, impairment in behavioral control, craving, diminished recognition of significant problems with a subject's behaviors and interpersonal relationships, and a dysfunctional emotional response. Like other chronic diseases, addiction often involves cycles of relapse and remission. Without treatment or engagement in recovery activities, addiction is progressive and can result in disability or premature death. Examples of addictive diseases or disorders include, but are not limited to, alcohol or drug abuse.

The term “alcoholism” according to the invention includes alcohol abuse, alcohol dependence and other problems with alcohol, and is generally used to mean compulsive and uncontrolled consumption of alcoholic beverages, usually to the detriment of the drinker's health, personal relationships, and social standing. It is a chronic disease, specifically an addictive illness.

The term “alcohol abuse” is defined in the Diagnostic and Statistical Manual of Mental Disorders (DSM-IV). Alcohol abuse as a maladaptive pattern of alcohol use that leads to clinically significant impairment or distress. Symptoms include one or more of the following occurring within a 12-month period: recurrent alcohol use that results in a failure to fulfill major role obligations at work, school or home; recurrent alcohol use in physically hazardous situations; recurrent alcohol-related legal problems; and continued alcohol use despite having persistent or recurrent social or interpersonal problems caused or exacerbated by the effects of the substance

By “alcohol dependence” is meant symptoms of alcohol abuse accompanied by one or more of the following: alcohol tolerance; withdrawal manifested by characteristic withdrawal syndrome for alcohol or alcohol taken to relieve or avoid withdrawal symptoms; alcohol taken in larger amounts over a longer period than as intended; a persistent desire or unsuccessful efforts to reduce or control drinking; much time spent in activities necessary to obtain alcohol, use alcohol, or recover from its effects; important social, occupational, or recreational activities being given up or reduced because of drinking; and continued use despite knowledge of having a persistent or recurrent physical or psychological problem caused or exacerbated by alcohol.

By “alcohol detoxification” or “detox” is meant the abrupt cessation of alcohol intake. The detox may be coupled with substitution of cross-tolerant drugs that have effects similar to the effects of alcohol in order to prevent severe alcohol withdrawal symptoms.

By “alcohol tolerance” is meant a need for markedly increased amounts of alcohol to achieve intoxication or desired effect or a markedly diminished effect with continued use of the same amount of alcohol.

By “alcohol withdrawal” is meant the symptoms associated with cessation of alcohol intake. With repeated heavy consumption of alcohol, gamma-aminobutyric acid receptors are desensitized and reduced in number, resulting in tolerance and physical dependence. When alcohol consumption is stopped too abruptly, the person's nervous system suffers from uncontrolled synapse firing. This can result in symptoms that include anxiety, life threatening seizures, delirium tremens, hallucinations, shakes and possible heart failure.

The term “treatment of alcoholism” comprises the amelioration, reduction or cessation of the desire for and habit of consuming alcoholic drinks, the treatment of alcohol dependence and the treatment of abstinence syndrome.

The term “excessive drinking” means consumption of alcohol for an extended period of time that results in intoxication that lasts longer than twice the time it would normally take the animal to reduce the amount of alcohol in the blood to below intoxicating levels.

The term “heavy drinking” means consumption of alcohol that result in rapid intoxication, or a rapid increase in alcohol concentration in the blood to levels that would normally result in intoxication.

By “talampanel” is meant a noncompetitive antagonist of the AMPA receptor, a type of glutamate receptor in the central nervous system, with a general structure of Structure I:

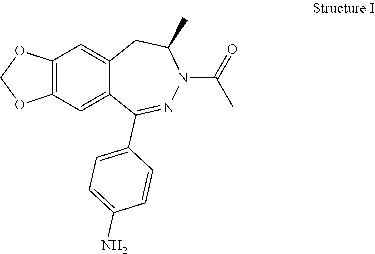 [SMILES: CC(=O)N1N=C(c2ccc(N)cc2)c2cc3c(cc2C[C@H]1C)OCO3]

By “perampanel” is meant a selective noncompetitive antagonist of AMPA receptors, the major subtype of ionotropic glutamate receptors, with a general structure of Structure II:

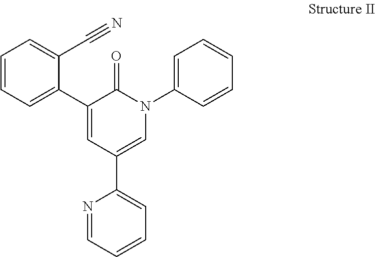 [SMILES: N#Cc1ccccc1-c1cc(-c2ccccn2)cn(-c2ccccc2)c1=O]

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “binder, excipient, diluent” is meant a non-active ingredient. Non-active ingredients may solubilize, suspend, thicken, dilute, emulsify, stabilize, preserve, protect, color, flavor, and/or fashion active ingredients into an applicable and efficacious preparation, such that it may be safe, convenient, and/or otherwise acceptable for use. Examples of excipients include, but are not limited to, solvents, carriers, diluents, binders, fillers, sweeteners, aromas, pH modifiers, viscosity modifiers, antioxidants, extenders, humectants, disintegrating agents, solution-retarding agents, absorption accelerators, wetting agents, absorbents, lubricants, coloring agents, dispersing agents, and preservatives. Excipients may have more than one role or function, or may be classified in more than one group; classifications are descriptive only and are not intended to be limiting. In some embodiments, for example, the at least one excipient may be chosen from corn starch, lactose, glucose, microcrystalline cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose, and fatty substances such as hard fat or suitable mixtures thereof.

A “dose” means the measured quantity of an active agent to be taken at one time by a patient.

A “dosage form” means a unit of administration of an active agent. Examples of dosage forms include tablets, capsules, injections, suspensions, liquids, emulsions, creams, ointments, suppositories, inhalable forms, transdermal forms, and the like.

By “effective amount” is meant the amount required to reduce or improve at least one symptom of a disease relative to an untreated patient. The effective amount of an active compound(s) used for therapeutic treatment of a disease varies depending upon the manner of administration, the age, body weight, and general health of the subject.

“Efficacy” means the ability of an active agent administered to a patient to produce a therapeutic effect in the patient.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutical composition” or “pharmaceutically acceptable composition” refers to a mixture of at least one compound or molecule useful within the invention with a pharmaceutically acceptable carrier. The pharmaceutical composition facilitates administration of the compound or molecule to a patient. Multiple techniques of administering a compound or molecule exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound or molecule useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

By “reference” is meant a standard or control. A “reference” is a defined standard or control used as a basis for comparison.

As used herein, “sample” or “biological sample” refers to anything, which may contain the cells of interest (e.g., neurons) for which the screening method or treatment is desired. The sample may be a biological sample, such as a biological fluid or a biological tissue. In one embodiment, a biological sample is a tissue sample including pulmonary arterial endothelial cells. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, the terms “treat,” treating,” “treatment,” and the like refer to decreasing, reducing or improving a disorder and/or symptom associated therewith. It will be appreciated that, although not precluded, treating a disorder or condition does not require that the disorder, condition or symptoms associated therewith be completely ameliorated or eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Compositions

It has been discovered that AMPA receptor (AMPA-Rs) antagonists (i.e., blockers), such as perampanel and talampanel, reduce drug and alcohol seeking behaviors in mammals. Activation of glutamatergic AMPA receptors promotes addictive behaviors in mammals. The invention includes compositions comprising talampanel and/or perampanel. The invention includes, in one aspect, compositions for treating or preventing an addictive disease or disorder, such as alcoholism, in a subject in need thereof comprising administering an effective amount of at least one of talampanel and perampanel.

Altered glutamatergic neurotransmission may play a significant role in the pathophysiology of alcoholism, and in the development of some of the more severe consequences of abrupt drinking cessation including delirium tremens and withdrawal seizures. Glutaminergic neurotransmission within and on dopaminergic pathways are thought to be a key and, likely necessary, component for the development and maintenance of addiction (Mol Psychiatry. 2003; 8(4):373-82).

Glutamatergic activation of ventral tegmental area dopaminergic neurons and subsequent dopamine release in the nucleus accumbens shell (NAS) is important in addiction, but activation of AMPA receptors presynaptically in the NAS and also activation of AMPA receptors in the prefrontal cortex (PFC) appears to potentiate dopaminergic neurotransmission (reward and positive reinforcement) in the NAS.

The role of AMPA receptors in addiction is still being elucidated, however it is clear that AMPA receptors likely play a significant role in addiction related neuroplasticity and learned behaviors. It has been discovered that AMPAR antagonists (such as talampanel and perampanel) have an opposite effect of the agonists, increasing the negative subjective experience of alcohol consumption and intoxication to produce a protective effect against excessive drinking. Topiramate, an anticonvulsant that is efficacious in treating alcohol dependence, has a multifaceted mechanism of action that includes potentiation of GABAergic neurotransmission, blockade of L-type calcium channels, in addition to antagonism of the kainite receptor to which it binds. Topiramate is also thought to block AMPA receptors as well although the evidence supporting this action in the literature is less clear. AMPA and kainite receptor antagonism may be related to the anti-addictive effects of topiramate.

Talampanel is a more selective and likely a stronger AMPAR antagonist than topiramate. Talampanel is an orally active noncompetitive (allosteric) antagonist of the AMPA receptor that is easily absorbed in the gastrointestinal system. It also demonstrates good blood-brain-barrier penetration. Animal model studies of animal spinal neurons have demonstrated the selective action of talampanel on the AMPA receptor, and preclinical studies have indicated a direct action on an allosteric site, referred to as the GYKI receptor, while not acting directly on the AMPA receptor itself.

Talampanel was developed as an anti-seizure medication from a series of novel 2,3-benzodiazepines which do not share the same pharmacologic profile or affinity for the benzodiazepine-GABA receptor complex that conventional 1,4-benzodiazepines do. In healthy human subjects, talampanel is well tolerated with a maximum tolerated single oral dose of 100 mg. Medication related adverse events/side effects have included dizziness, sedation, and ataxia. These adverse events/side effects have been related to plasma talampanel levels with the majority of events occurring at oral doses greater than or equal to. Pharmacokinetically, talampanel has an elimination half-life T_{1/2} of approximately 4 hours in healthy volunteers and reaches peak plasma concentration (Tₘₐₓ) approximately 2 hours post-ingestion.

Like talampanel, perampanel is a noncompetitive (allosteric) antagonist of the AMPA-R that has about 100% bioavailability, effectively penetrates the blood-brain-barrier, and rapidly reaches peak plasma concentrations (˜1 hour). Perampanel is FDA approved for the adjunctive treatment of refractory partial-onset seizures. Phase III trials in epilepsy have demonstrated that perampanel is very well tolerated, and safe for administration. In vitro studies of rat hippocampal slices have demonstrated the selective action of perampanel on the AMPA receptor. Similar to talampanel, perampanel does not appear to block the ion channel, but rather binds the AMPA-R at the GYKI receptor.

In human subjects, perampanel is well tolerated at doses of 8 mg or 12 mg daily, with titration helpful to reach higher doses for ongoing therapy. Medication related adverse events/side effects with chronic dosing in epilepsy trials have included; dizziness, somnolence, irritability, headache, fall, and ataxia. Pharmacokinetically, perampanel has an elimination half-life T_{1/2} of over 52 hours (estimated at 70 hours, but with a wide range from 52-129 hours) and reaches peak plasma concentration (Tₘₐₓ) approximately 1 hour post-ingestion. Perampanel is primarily metabolized by CYP3A4 and exhibits linear pharmacokinetics.

In one aspect, the invention includes a composition for treating or preventing an addictive disease or disorder, comprising an effective amount of at least one of talampanel and perampanel.

The invention includes a pharmaceutical composition. Such a pharmaceutical composition may includes a pharmaceutically acceptable carrier or adjuvant. The composition may be provided in a form suitable for administration to a subject, and may be comprise one or more pharmaceutically acceptable carriers, one or more additional ingredients, or some combination of these. The composition of the invention may comprise a physiologically acceptable salt, such as a compound contemplated within the invention in combination with a physiologically acceptable cation or anion, as is well known in the art.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human patient being treated, and the like. In one embodiment, the composition is formulated for oral administration, such as a liquid suspension, a chewable composition, and an orally disintegrating tablet or capsule composition.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In one embodiment, the compositions of the invention are formulated using at least one pharmaceutically acceptable binder, excipient, diluent, or any combinations thereof. In one embodiment, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The compound or a combination of compounds for the treatment of an addictive disease or disorder may be prepared by any suitable means that results in a concentration of the therapeutic that, combined with other components, is effective in treating, decreasing, reducing, or stabilizing the addictive disease or disorder. In one embodiment, a composition of the invention comprises talampanel and/or perampanel. In another embodiment, a composition of the invention comprises a cell contacted with talampanel and/or perampanel. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice, as a skilled artisan recognizes it is routine in the art to modify the dosage for humans compared to animal models. In certain embodiments it is envisioned that the dosage may include an effective amount from between about 0.001 mg compound/Kg body weight to about 100 mg compound/Kg body weight; or from about 0.05 mg/Kg body weight to about 75 mg/Kg body weight or from about 0.1 mg/Kg body weight to about 50 mg/Kg body weight; or from about 0.5 mg/Kg body weight to about 40 mg/Kg body weight; or from about 0.1 mg/Kg body weight to about 30 mg/Kg body weight; or from about 1 mg/Kg body weight to about 20 mg/Kg body weight. In other embodiments, the effective amount may be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, or about 100 mg/Kg body weight. In other embodiments, it is envisaged that effective amounts may be in the range of about 2 mg compound to about 100 mg compound. In other embodiments, the effective amount may be about 1, 2, 3, 4, 5, 6, 7, 8, 9, 10, 11, 12, 13, 14, 15, 16, 17, 18, 19, or 20 mg per single dose. In another embodiment, the effective amount comprises less than about 2, 3, 4, 5, 6, 7, 8, 9, 10, 15, 20, 25, 30, 35, 40, 45, 50, 55, 60, 65, 70, 75, 80, 85, 90, 95 mg daily. In an exemplary embodiment, the effective amount comprises less than about 50 mg daily. Of course, the single dosage amount or daily dosage amount may be adjusted upward or downward, as is routinely done in such treatment protocols, depending on the results of the initial clinical trials and the needs of a particular patient.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration of a composition of the invention to a suitable animal model to identify the dosage of the composition(s), concentration of components therein and timing of administering the composition(s), which elicit tissue repair, reduce cell death, or induce another desirable biological response. Such determinations do not require undue experimentation, but are routine and can be ascertained without undue experimentation.

The biologically active agents can be conveniently provided to a subject as sterile liquid preparations, e.g., isotonic aqueous solutions, suspensions, emulsions, dispersions, or viscous compositions, which may be buffered to a selected pH. Cells and agents of the invention may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by suspending talampanel and/or perampanel in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

### Treatment

In one aspect, the invention includes a method of treating or preventing an addictive disease or disorder in a subject in need thereof comprising administering an effective amount of at least one of talampanel and perampanel.

The addictive disease or disorder includes alcoholism, alcohol dependence, or alcohol withdrawal, heavy alcohol consumption, excessive alcohol consumption, and combinations thereof.

In one embodiment, the method includes decreasing frequency of alcohol consumption. In another embodiment, the method includes decreasing alcohol consumption compared with before administration. In yet another embodiment, the method includes decreasing alcohol consumption and increasing abstinence of alcohol consumption.

The method also includes administering an effective amount of the talampanel and perampanel. The administration can include about 0.05 mg to about 0.5 g per single dose, less than about 0.80 g daily, or a range of about 0.001 mg/kg to about 100 mg/kg. The administration can also be via an oral route.

In another aspect, the invention includes a composition and method of treating or preventing an addictive disease or disorder, comprising an effective amount of at least one of talampanel and perampanel, wherein the addictive disease or disorder is selected from the group consisting of alcoholism, alcohol dependence, or alcohol withdrawal, heavy alcohol consumption, excessive alcohol consumption, and combinations thereof.

In yet another aspect, the invention includes a method of decreasing alcohol consumption in a subject in need thereof comprising administering an effective amount of at least one of talampanel and perampanel.

### Methods of Delivery

Compositions comprising talampanel and/or perampanel may be delivered to a subject in need thereof. Modes of administration include intramuscular, intra-cardiac, intra-hepatic, oral, rectal, topical, intraocular, buccal, intravaginal, intracisternal, intra-arterial, intracerebroventricular, intratracheal, nasal, transdermal, within/on implants, e.g., fibers such as collagen, osmotic pumps, or parenteral routes. The term “parenteral” includes subcutaneous, intravenous, intramuscular, intraperitoneal, intragonadal or infusion.

The compositions can be administered via localized injection, including catheter administration, systemic injection, localized injection, intravenous injection, or parenteral administration. When administering a therapeutic composition of the present invention, it will generally be formulated in a unit dosage injectable form (solution, suspension, emulsion). Dosages can be readily adjusted by those skilled in the art (e.g., a decrease in purity may require an increase in dosage). Compositions of the invention can be introduced by injection, catheter, or the like. Compositions of the invention include pharmaceutical compositions comprising cellular factors of the invention and a pharmaceutically acceptable carrier. Administration can be autologous or heterologous.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a certain cell or tissue by using carriers or chemical derivatives to deliver the therapeutic agent to a particular cell type (e.g., neuron). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active agent). The coating may be applied on the solid dosage form in a similar manner as that described in Encyclopedia of Pharmaceutical Technology, supra.

At least two active agents may be mixed together in the tablet, or may be partitioned. In one example, the first active agent is contained on the inside of the tablet, and the second active agent is on the outside, such that a substantial portion of the second active agent is released prior to the release of the first active agent.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Delayed Release Oral Dosage Forms

In one embodiment, the composition is formulated for delayed-release.

Delayed release can included controlled release compositions for oral use, e.g., constructed to release the active agent by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydro gels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates the addictive disease or disorder, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactia poly-(isobutyl cyanoacrylate), poly(2-hydroxyethyl-L-glutam-nine) and, poly(lactic acid).

Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out embodiments of the present invention, and are not to be construed as limiting in any way.

The Materials and Methods used in the performance of the experiments disclosed herein are now described.

Subjects/Recruitment:

Standard NIAAA alcohol administration study recruitment and safety guidelines will be followed to determine patients' eligibility for participation. Participants will consist of fifty NTSHD (N=50). NTSHD status will be based on scores on the Alcohol Use Disorders Identification Test (AUDIT), which define hazardous drinkers as those exceeding gender specific weekly limits. To meet criteria for NTSHD, subjects will have a score of 8 or more on the alcohol use disorders identification test (AUDIT), and drink more than 4 standard drinks (SD) per day on at least one day per week on average for men, and >3 SD/day at least once weekly for women. The NTSHD population has been used in prior alcohol administration studies (Alcohol Clin Exp Res. 2000; 24(6):789-94). Baseline drinking, and drinking in between the lab administration sessions will be evaluated using the Timeline Follow Back Method (TLFB). The study plan will be to enter 50 subjects in the sessions, approximately 35 male and 15 female, with the sample inclusive of minorities and representative of the greater New Haven County. Participants will be recruited through advertisement in the community. Up to 100 subjects will be recruited and screened, with the goal to enter 50 subjects and to complete 42 subjects. Subjects will earn $150 for the first session, $150 for the second, and $150 for the third laboratory session. Subjects can earn up to an additional $90 from the study based on their scoring on one of the tasks in the study, the Balloon Analogue Risk Task. Subjects will also receive $20 for completing the screening process.

The inclusion criteria will be: 1) males and females 2) between the ages of 21 and 55 years; 3) NTSHDs as defined above, and must have had at least 5 SD in one day on at least some occasions in the past and been able to tolerate it without an adverse reaction 4) generally medically and neurologically healthy on the basis of history, physical examination, EKG, screening laboratory results (CBC w/differential, TSH, Free-T4, AST, ALT, GGT, BUN, creatinine, electrolytes, urinalysis, beta-HCG). Individuals with LFTs that are no more than 3 times above the normal levels will be included; 5) women with a negative pregnancy test and not nursing, must be regularly using birth control 6) negative breath alcohol at screening and on each test day; 7) not taking any psychoactive medication or opioids (in past 30-days); and 8) are non-treatment seeking. Exclusion criteria: 1) they need detoxification determined by a CIWA score of >8 or have had a history of alcohol detoxification in the past; 2) have been in treatment for an alcohol problem within the last 6 months, or if the severity of their alcohol problem based on the research physician's assessment warrants definitive treatment; 3) meet criteria for DSM-IV psychiatric and substance use disorder diagnosis (other than alcohol abuse/dependence, and nicotine dependence; those diagnoses will be allowed; participants can be either smokers up to 1 pack per day or non-smokers) based on history and psychiatric evaluation that includes a structured diagnostic interview (Structured Clinical Interview for DSM-IV Axis I Disorders: SCID); 4) unwillingness to remain alcohol-free 12 hours prior to test days; 5) have a significant ongoing serious medical condition such as Diabetes Mellitus, liver disease (see above LFT guideline), renal disease (as evidenced by serum creatinine above our laboratory's reference limit of 1.7 mg/dL, or have a history of adverse reaction to IV placement/blood draw.

Laboratory Session Overview:

Participants will complete 3 test days each, 2 weeks apart, in a randomized counter-balanced order, under double-blind conditions, following either pretreatment with daily perampanel 2 mg or placebo 7 days prior to each test day, and then observed dosing (of placebo, moderate 6 mg dose perampanel, or high dose 10 mg) in the lab 1 hour before alcohol infusion (a one time dose). Each lab session occurs exactly a week after starting the medication for that round, which means they will have taken it for 7 days prior, and the lab is the eighth day in a row on medication. The wash out period between lab test session phases will be 7-10 days. Subjects will receive 2 days of 2 mg perampanel after each lab to taper down (included in the washout period). The next appointment after a session will be a brief one at the start of the next phase (see day 1 of the phases in Table 1 below), at which point the next week of low dose perampanel or placebo will be started). With the washout period (7-10 days) and the 7 day taper of the next phase, the actual lab sessions will occur 14-17 days apart. All test days will involve administration of alcohol with the same 3 target doses (target BrAc=20 mg %, BrAc=60 mg %, and BrAc=100 mg %) in a step-wise fashion. Phone contact with subjects will be maintained during the week that they are on medication and will talk to them once by phone in days 2-4 on the medication, and once between days 5-7, inquiring about side effects, especially neuropsychiatric side effects. If there is a concern, the subjects will be asked to come to the research center for evaluation by study staff and/or a study physician as needed. They will be able to contact the Biostudies on-call personnel at any time. After the screening visits, which will take place most likely on 2 separate days (1-2 visits), subjects will begin the medication and laboratory administration phases, (see below schematic).

TABLE 1
Schematic Overview

| Phase I | | | Phase II | | | Phase III | | |
|---|---|---|---|---|---|---|---|---|
| Day 1 | Day 8 | Washout | Day 1 | Day 8 | Washout | Day 1 | Day 8 | Finish |
| Outpatient | Lab | for 7-10 | Outpatient | Lab | for 7-10 | Outpatient | Lab | Includes 2 |
| Visit: | Session: | days: | Visit: | Session: | days: | Visit: | Session: | days of |
| Start PRM | PRM | Includes 2 | Start PRM | PRM | Includes 2 | Start PRM | PRM | 2 mg PRM |
| 2 mg daily | 6 mg or | days of | 2 mg daily | 6 mg or | days of | 2 mg daily | 6 mg or | or PLC |
| or PLC | 10 mg, | 2 mg PRM | or PLC | 10 mg, | 2 mg PRM | or PLC | 10 mg, or | taper |
| | or PLC | or PLC | | or PLC | or PLC | | PLC | |
| | | taper | | | taper | | | |

PRM = Perampanel,
PLC = Placebo

Screening and Initial Assessments:

After providing written informed consent, subjects will be interviewed with the SCID. The AUDIT (World Health Organization; 1989) and the timeline follow-back (TLFB) (Humana Press; 1992. p. 41-72) will be used to document heavy drinking status and the degree of daily alcohol consumption prior to entry. Subjects will have a psychiatric/medical examination by a study psychiatrist. Family history of alcohol problems will be evaluated using the family history assessment module (FHAM). Trait impulsivity will be measured with the Barrett Impulsivity Scale (BIS). The screening and initial assessments will likely be completed in 1-2 visits.

Experimental Procedure:

After initial screening, eligible participants will be randomized to receive double-blind perampanel (moderate and high dose) or placebo in a counterbalanced order (i.e., each subject will receive all three conditions; perampanel moderate dose, high dose, and placebo, but in a random and counterbalanced order). Perampanel and placebo will be packaged in identical looking capsules. The research pharmacist will perform randomization. Study staff will be blinded during the study to medication/order assignment. All subjects will be informed of receiving alcohol and how much (peak BrAC) on both occasions but will be kept blind to the real-time BrAC levels during the lab sessions. Participants will be asked to fast after midnight the night before, and come to the laboratory at approximately 8:00 A.M on test days. Urine for toxicology screen, pregnancy screen, and breath alcohol level, will be assessed at screening and on each test day. At the beginning of each test day, and before a standardized breakfast, an IV line will be inserted, a blood sample will be taken for perampanel trough levels, then participants will take their 6 mg, 10 mg perampanel, or placebo dose in the lab. In order to maximize perampanel absorption, breakfast will be given 30 minutes after the medication is taken. One hour after ingesting perampanel, infusion of alcohol begins.

Alcohol will be administered IV for 10 minutes to achieve the first target BrAc level of 20 mg %. Once the first BrAc is achieved, it will be maintained using a clamp procedure for 30 minutes for measurements (outcomes) to be obtained. This will be followed by additional administration of alcohol to achieve BrAc level of 60 mg % for 10 minutes. This alcohol level will be maintained for 30 minutes. The last phase of the alcohol administration will consist of alcohol infusion to reach BrAc level of 100 mg %, over 10 minutes, and this level will be maintained for 30 minutes. The subjects will remain on the Biostudies Unit for several hours until the effects of alcohol have worn off. A physician will clear subjects at the end of each test day. Subjects must have a BrAC of <20 mg % before leaving the unit. The 3 step increasing IV ethanol dose paradigm is a reliable and valid method. (Curr Pharm Des. 2010; 16(19):2149-58) See Table 2.

If subjects do not present with a BrAC of 0, or if the drug screen is positive for drugs of abuse, a decision will be made by study staff and the PI about whether to end the subjects participation or to allow them to reschedule for some point over the next 7 days. The 2 mg daily perampanel would be extended for up to 7 days in the case of rescheduling.

Alcohol Solution:

The IV alcohol solution is a 6% alcohol solution in 0.9% saline, with approximately 2 oz. of ethanol in each bag. Two IV solution bags will be used with a dual “Gemini” pump controlled by a computerized assisted infusion system that adjusts for optimal alcohol administration based on personalized parameters.

Behavioral Assessments:

On experimental session days (after the pretreatment with perampanel or placebo), participants will complete self-report measures of recent substance/alcohol use. Smokers will complete the Fagerstrom Test for Nicotine Dependence [FTND] at baseline, and will complete the Minnesota Nicotine Withdrawal Scale along other measures during lab sessions, in order to measure and control for nicotine withdrawal effects. Subjects will be asked to have their last cigarette prior to starting pre-lab medication administration and activities, at each session.

During the Ethanol Infusion Sessions:

measures of subjective responses to alcohol activation, stimulation, and craving (“the alcohol effects battery”), will be administered at baseline and at each of the BrAC levels 0.00, 0.02, 0.06, and 0.10 g/dL (see Table 2):

TABLE 2
Experimental Session Day

| Time | |
|---|---|
| (minutes) | Assessments |
| T−65 | Urine drug screen, breathalyzer, IV line inserted, perampanel blood level |
| | drawn |
| T−60 | Take oral study medication (either perampanel or placebo), Vital Signs, |
| T−30 | Pre-alcohol challenge measures, Standardized breakfast |
| T−10 | Alcohol-effects battery (BAES, DEQ, VAS, AUQ, MNWS, SEQ, SHAS, |
| | POMS, SST, BART) at BrAC 0.00 g/dL |
| 0 | Infusion begins, Vital Signs |
| T+10 | Infusion clamped (approximately 30 min) alcohol BrAC 0.02 g/dL |
| T+15 | Alcohol-effects assessment battery (BAES, DEQ, VAS, AUQ, MNWS, SEQ, |
| | SHAS, POMS, SST, BART) |
| T+40 | Infusion increased to target next alcohol level, Vital Signs |
| T+50 | Infusion clamped (approximately 30 min) alcohol BrAC 0.06 g/dL |
| T+55 | Alcohol-effects assessment battery (BAES, DEQ, VAS, AUQ, MNWS, SEQ, |
| | SHAS, POMS, SST, BART) |
| T+80 | Infusion increased to target next alcohol level, Vital Signs |
| T+90 | Infusion clamped (approximately 30 min) alcohol BrAC 0.10 g/dL |
| T+95 | Alcohol-effects assessment battery (BAES, DEQ, VAS, AUQ, MNWS, SEQ, |
| | SHAS, POMS, SST, BART), |
| T+125 | Infusion ends |
| T+240 | Test day debriefing and discharge when BrAC <0.02 g/dL |

Biphasic Alcohol Affects Scale (BAES):

A 14-item scale with 7 items designed to assess stimulant effects from alcohol intoxication and 7 items developed to measure the sedative effects of alcohol. This scale was selected as a primary outcome measure because it is sensitive to the effect of alcohol.

Drug Effects Questionnaire (DEQ):

consists of four items that measure current alcohol effects: ‘feel alcohol’, ‘feel high’, ‘like alcohol’, and ‘want more alcohol’.

Visual Analog Scales of Mood States (VAS):

A 5-item scale designed to assess subjective alcohol effects: high, anxious, drowsy, irritable, and nauseous. It has been used in previous studies by our group and has good sensitivity to drug effect.

Alcohol Urge Questionnaire (AUQ):

A valid eight-item Likert-type scale designed to assess acute alcohol craving.

Subjective High Assessment Scale (SHAS):

We will use the SHAS7, a reliable and valid 7-item scale assessing the amount (ranging from 0-36) of each experiential item (e.g., feeling “drunk” or “high”) related to alcohol exposure.

Side Effect Questionnaire (SEQ):

This consists of a list of side effects associated with perampanel (e.g., fatigue, dizziness), rated from 0=“none” to 4=“severe.”

POMS 2 Short Versions:

The POMS 2 short version contains a subset of 35 items from the full-length versions. This subset comprises those five items on full version POMS scale that exhibited good item-total correlations and best predicted their respective scale scores.

Stop Signal Test (SST, Also Know as go/No-go):

SST is a commonly used test of response inhibition. This test consists of two parts. In the first part, the subject is introduced to the press pad, and told to press the left hand button when they see a left-pointing arrow or press the right hand button when they see a right-pointing arrow. There is one block of 16 trials for the subject to practice this. In the second part, the subject is told to continue pressing the buttons on the press pad when they see the arrows, as before, but, if they hear an auditory signal (a beep), they should withhold their response and not press the button.

The Balloon Analogue Risk Task (BART):

This is a valid and commonly used test of risk-taking, an aspect of impulsivity. This task is computerized, and subjects press a button to analogously pump air into a balloon on the screen. Subjects are told they can earn 1 cent for each pump of the balloon banked to a reward account before they cash out and stop pumping. The balloons break at various random intervals and if the balloon breaks, the subjects lose money out of the reward account. We will use a 10-balloon paradigm, a briefer but valid version of this task. They can earn a maximum of 7.5 dollars per administration, with 4 administrations per lab, for a total of up to $90 additional pay over the course of the study, though actual payouts will likely be substantially less.

Data Analysis:

We have well developed statistical methods to analyze alcohol challenge studies. All statistical testing will be at a two-tailed alpha level of 0.05 and will be performed in SAS. For the primary, and secondary hypotheses we will use mixed-effects models to assess change in stimulation, sedation, and craving for alcohol during intravenous infusion of alcohol. Pretreatment medication (perampanel moderate versus high dose versus placebo) and gender will be used as between-subject factors, and alcohol concentration doses (target BrACs of 0.00, 0.02, 0.06, and 0.10 g/dL) as a four-level within-subject factor. The primary goal of these analyses will be to test for differences in measures of stimulation, enjoyment, craving, and sensitivity to alcohol as a function of perampanel pretreatment. The primary outcome dependent variable will be stimulation as measured by BAES. We will explore the measure of trait impulsivity (BIS) as a covariate, but will also explore the effect of genotype in a mixed model analysis as in independent variable in a pharmacogenetic model. We will explore the effects of perampanel, alcohol, and genotype on measures of state impulsivity and risk taking.

Power Analysis:

No human studies have examined perampanel in psychiatric/substance-using populations. We calculated an effect size for naltrexone based on Ray et al. (Arch Gen Psychiatry. 2007; 64(9):1069-77), for the effect of naltrexone on stimulation (BAES) and will extrapolate this effect to perampanel. Based on this calculated effect size (d) of 0.45 (e.g. a moderate effect size), we should be able to show a significant difference between perampanel and placebo groups using a two-sided, paired sample t-test, with alpha=0.05 and power (1-Beta) of 0.80 with n=21 subjects in each experimental group (total N=42). We will recruit and enter N=50 subjects, in order to complete at least 42.

All data will be stored without direct identifiable information, but will be identifiable via a linking code. Any hard copy records associated with the study will be kept in locked offices in our VA alcohol clinical trials unit. The secured research records are labeled with code numbers only (names and other identifying information are kept separate from research records). Access to hard copy data is only given to staff members working on the study. Only staff members designated to handle or analyze study samples will have access to the samples and their storage. As per routine in the VACT, all electronic files (e.g., database, spreadsheet) will be password protected. Any computer hosting such files will have a BIOS password to prevent access by un-authorized users. Furthermore, for systems not running Windows 2000/XP or later versions, a password-protected screen saver will be installed and configured to activate ten minutes after the computer has been idle.

Data used for safety monitoring will include serious adverse events, dropout rates and reasons for dropout, enrollment numbers, subject interviews, medication compliance, review of symptoms or performance status, review of clinical/diagnostic test results, review of physical examination, review of vital signs, review of evaluation performed, protocol deviations, and blinded data. If it has been determined, for any reason, that there will be a suspension of this study, the PI will suspend enrollment of new subjects but continue intervention/monitoring of previously enrolled subjects if it is in the best interest of those subjects. This study involves only a greater than minimal risk to subjects, and that potential benefits outweigh potential risks of participation.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating or preventing an addictive disease or disorder in a subject in need thereof comprising administering an effective amount of at least one of talampanel and perampanel.

2. The method of claim 1, wherein the addictive disease or disorder comprises heavy alcohol consumption or excessive alcohol consumption.

3. The method of claim 1, wherein the addictive disease or disorder is alcoholism, alcohol dependence, or alcohol withdrawal.

4. The method of claim 3, wherein the step of administrating results in decreasing frequency of alcohol consumption.

5. The method of claim 1, wherein the step of administrating results in decreasing alcohol consumption compared with before administration.

6. The method of claim 1, wherein the step of administrating results in decreasing alcohol consumption and increasing abstinence of alcohol consumption.

7. The method of claim 1, wherein the step of administering the effective amount comprises administering about 0.05 mg to about 0.5 g per single dose.

8. The method of claim 1, wherein the step of administering the effective amount comprises administering less than about 0.80 g daily.

9. The method of claim 1, wherein the effective amount administered to the subject is within the range of about 0.001 mg/kg to about 100 mg/kg.

10. The method of claim 1, wherein the step of administering comprises administration via an oral route.

11. A method of decreasing alcohol consumption in a subject in need thereof comprising administering an effective amount of at least one of talampanel and perampanel.

12. A composition for treating or preventing an addictive disease or disorder, comprising an effective amount of at least one of talampanel and perampanel.

13. The composition of claim 12 further comprising a pharmaceutically acceptable carrier or adjuvant.

14. The composition of claim 12, wherein the composition is formulated for oral administration.

15. The composition of claim 14, wherein the composition is selected from the group consisting of a liquid suspension, a chewable composition, and an orally disintegrating tablet or capsule composition.

16. The composition of claim 14, wherein the composition is formulated for delayed-release.

17. The composition of claim 12, wherein the effective amount comprises about 2 mg to about 100 mg per single dose.

18. The composition of claim 12, wherein the effective amount comprises less than about 50 mg daily.

19. The composition of claim 12, wherein the effective amount administered to the subject is within the range of about 0.001 mg/kg to about 100 mg/kg.

20. The composition of claim 12 further comprising at least one binder, excipient, diluent, or any combinations thereof.

21. A composition for treating or preventing an addictive disease or disorder, comprising an effective amount of at least one of talampanel and perampanel, wherein the addictive disease or disorder is selected from the group consisting of alcoholism, alcohol dependence, or alcohol withdrawal, heavy alcohol consumption, excessive alcohol consumption, and combinations thereof.
